(19) United States
(12) Patent Application Publication
(10) Pub. No.: US 2006/0040354 A1
(43) Pub. Date: Feb. 23, 2006

(54) Novel polyadenylation signal for use in expression vectors

(76) Inventor: Theresa L. O'Keefe, Waltham, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/185,436

(22) Filed: Jul. 20, 2005

Related U.S. Application Data
(60) Provisional application No. 60/589,679, filed on Jul. 20, 2004.

(51) Int. Cl. C07K 16/44; C07H 21/04; C12P 21/06; C12N 5/06

(52) U.S. Cl. 435/69.1; 435/320.1; 435/328; 530/387.3; 536/23.53

(57) ABSTRACT

Disclosed are novel polynucleotides and expression vectors which comprise an immunoglobulin gene polyadenylation signal and a DNA sequence encoding a polypeptide wherein the DNA sequence is other than a genomic DNA sequence from the immunoglobulin gene. Also, disclosed is a eukaryotic cell or cell-line comprising the novel expression vectors and a method of producing a desired polypeptide comprising culturing mammalian cells transfected with the novel expression vectors and recovering the desired polypeptide from the culture medium. The novel polynucleotides and expression vectors may comprise an immunoglobulin gene polyadenylation signal from an IgG1 heavy-chain gene.

### RELATED APPLICATION

This application claims the benefit of U.S. Provisional Application No. 60/589,679, filed on Jul. 20, 2004, the entire teachings of which are incorporated herein by reference.

### BACKGROUND OF THE INVENTION

The use of expression systems is an important tool for the production of recombinant proteins. Recombinant proteins have been successfully produced in both prokaryotic expression systems (such as E. coli) and eukaryotic expression systems (including yeast and mammalian systems) and can have utility as research and/or therapeutic agents. When producing recombinant proteins for therapeutic use, it is often preferred to use mammalian expression systems as these systems are more likely to produce proteins with essential post-translational modifications. The use of mammalian expression systems is, however, associated with a significant shortcoming in that the level of protein expression is often significantly less than that of corresponding prokaryotic expression systems. Several methods have been employed to obtain higher levels of recombinant gene expression; one such method pertains to the use and selection of a polyadenylation signal in the protein construct.

Polyadenylation signals are nucleotide sequences found in nearly all mammalian genes and control the addition of a string of approximately 200 adenosine residues (the poly(A) tail) to the 3′ end of the gene transcript (Proudfoot et al., Cell 108: 501-512 (2002)). The function of the poly(A) tail is not fully understood but its presence in mRNA is believed to be essential to mRNA stability as there is evidence that mRNAs lacking the poly(A) tail are rapidly degraded. There is also evidence that the presence of the poly(A) tail positively contributes to the translatability of mRNA by affecting the initiation of translation (Molecular Biology of the Cell, Third Edition by B. Alberts et al., Garland Publishing, 1994).

The polyadenylation signal is that portion of the RNA (and can also refer to the corresponding portion of DNA) that directs binding of the polyadenylation protein complex to mRNA. It is believed that efficient polyadenylation signals comprise at least two regions: the hexanucleotide sequence, AAUAAA, and either a downstream G/U or simply U-rich region. The AAUAAA sequence is believed to bind cleavage and polyadenylation specificity factor (CPSF) and the CPSF-RNA complex then binds additional proteins, including cleavage stimulatory factor (CStF) and poly(A) polymerase which are involved in cleavage of the mRNA and addition of the poly(A) tail. The interaction between CStF and the U-rich region is believed to contribute to the stability of the protein complex. Cleavage and polyadenylation ultimately take place 10-35 nucleotides downstream of the AAUAAA sequence.

Several polyadenylation signals have been utilized for recombinant protein expression and include polyadenylation signals from simian virus 40 (SV40) (Schek et al., Mol. Cell Biol. 12(12): 5386-93 (1992)), α-globin (Thein et al., Blood 71(2): 313-19), β-globin (Orkin et al., EMBO J. 4(2): 453-6 (1985)), human collagen, polyoma virus (Batt et al., Mol. Cell Biol. 15(9): 4783-90 (1995)) and bovine growth hormone (bGH) (Woychik et al., Proc. Natl. Acad. Sci. USA 81(13): 3944-8 (1984); U.S. Pat. No. 5,122,458). The available polyadenylation signals differ with respect to their effect on the level of protein expression in different cells systems. Although the bGH polyadenylation signal has been shown to result in higher protein expression when compared with other known polyadenylation signals in specific mammalian cells (Pfarr et al., DNA 5(2): 115-22 (1986); Yew et al., Hum. Gen. Ther. 8(5): 575-841 (1997)), there remains a need in the art for the identification and use of new polyadenylation signals which can contribute to efficient protein expression.

### SUMMARY OF THE INVENTION

It has now been discovered that high protein expression can be achieved using a polyadenylation signal from an immunoglobulin gene. For example, it has been shown that Chinese hamster ovary cell transfectants comprising polyadenylation signal sequences from the human IgG1 gene expressed high levels of recombinant protein (see Example 6 below).

The present invention pertains to novel polynucleotides and expression vectors comprising a polyadenylation signal from an immunoglobulin gene, cells comprising the novel expression vectors and novel methods for producing recombinant polypeptides in mammalian cells. In one embodiment, the invention pertains to a novel polynucleotide comprising a polyadenylation signal from an IgG gene. In another embodiment, the invention pertains to a novel expression vector comprising a polyadenylation signal from an IgG gene. In further embodiments, the polyadenylation signal is an IgG heavy-chain gene polyadenylation signal.

One embodiment of the present invention is a polynucleotide comprising an IgG heavy-chain gene polyadenylation signal and a DNA sequence encoding a polypeptide. The DNA sequence encoding a polypeptide is other than a genomic DNA sequence from the IgG heavy-chain gene. In addition, the DNA sequence is upstream from, and operably linked, to the IgG heavy-chain gene polyadenylation signal. Another embodiment of the present invention is a novel expression vector comprising an IgG heavy-chain gene polyadenylation signal and a DNA sequence encoding a polypeptide. The DNA sequence encoding a polypeptide is other than a genomic DNA sequence from the IgG heavy-chain gene. In addition, the DNA sequence is upstream from, and operably linked, to the IgG heavy-chain gene polyadenylation signal.

In another embodiment of the present invention, the IgG heavy chain gene polyadenylation signal is a human IgG heavy-chain gene polyadenylation signal. In a further embodiment, the human IgG heavy chain gene polyadenylation signal is an IgG1 heavy-chain, an IgG2 heavy-chain, an IgG3 heavy-chain or an IgG4 heavy-chain gene polyadenylation signal. In a further embodiment, the human IgG heavy-chain gene polyadenylation signal is an IgG1 heavy-chain gene polyadenylation signal. In an additional embodiment, the human IgG1 heavy-chain gene polyadenylation signal comprises a sequence selected from the group consisting of SEQ ID NO:10 and SEQ ID NO:9. In yet another embodiment, the human IgG1 heavy-chain gene polyadenylation signal consists of a sequence selected from the group consisting of SEQ ID NO:10 and SEQ ID NO:9.

Another embodiment of the present invention is a eukaryotic cell comprising the novel expression vector. In a further embodiment, the eukaryotic cell is a mammalian cell. Yet another embodiment of the present invention is a method for producing a desired polypeptide comprising culturing a mammalian cell transfected with the novel expression vector and recovering the desired polypeptide from the culture medium. In certain embodiments, the desired polypeptide is an immunoglobulin fusion protein. In certain other embodiments, the desired polypeptide is a high mobility group protein.

### BRIEF DESCRIPTION OF THE DRAWINGS

FIG. 1A is the nucleic acid sequence of the human IgG1 heavy-chain gene (SEQ ID NO: 1).

FIG. 1B is the nucleic acid sequence of the human IgG2 heavy-chain gene (SEQ ID NO: 2).

FIG. 1C is the nucleic acid sequence of the human IgG3 heavy-chain gene (SEQ ID NO: 3).

FIG. 1D is the nucleic acid sequence of the human IgG4 heavy-chain gene (SEQ ID NO: 4).

FIG. 2A is the nucleic acid sequence of the mouse IgG1 heavy-chain gene (SEQ ID NO: 5).

FIG. 2B is the nucleic acid sequence of the mouse IgG2a heavy-chain gene (SEQ ID NO: 6).

FIG. 2C is the nucleic acid sequence of the mouse IgG2b heavy-chain gene (SEQ ID NO: 7).

FIG. 2D is the nucleic acid sequence of the mouse IgG3 heavy-chain gene (SEQ ID NO: 8).

FIG. 3 is the nucleic acid sequence of human IgG1 heavy-chain gene polyadenylation signal sequence 1 (“IGPA”) (SEQ ID NO: 9).

FIG. 4 is the nucleic acid sequence of human IgG1 heavy-chain gene polyadenylation signal sequence 2 (“LIGPA”) (SEQ ID NO: 10).

FIG. 5A is the nucleic acid sequence of RAGE-Ig fusion protein.

FIG. 5B is the amino acid sequence of the RAGE-Ig fusion protein.

FIG. 6 is a diagram depicting the construction of IGPA and LIGPA.

FIG. 7A is a diagram showing the construction of mammalian expression vector pCTiTOK18, described in Example 3.

FIG. 7B is a diagram showing the construction of mammalian expression vector pCTiTOK28, described in Example 3.

FIG. 7C is a diagram showing mammalian expression vector pCTiTOK29, described in Example 3.

FIG. 8 is a graph comparing quantity of RAGE-immunoglobulin fusion protein (ug/ml) produced by CHO cells transfected with the vectors comprising the BGH, IGPA and LIGPA polyadenylation signal sequences.

FIG. 9 is a graph comparing the quantity of RAGE-immunoglobulin fusion protein (ug/ml) in the supernatants of different CHO cell clones transfected with the mammalian expression vector pCTiTOK29.

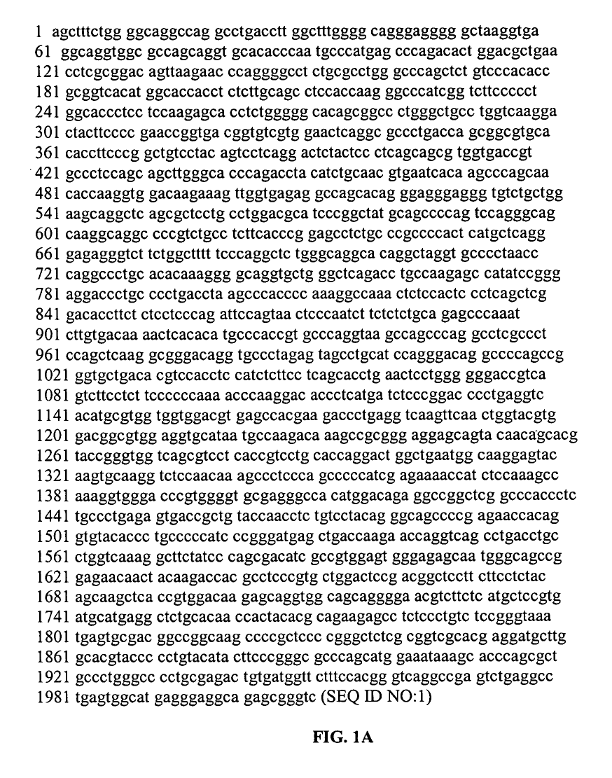

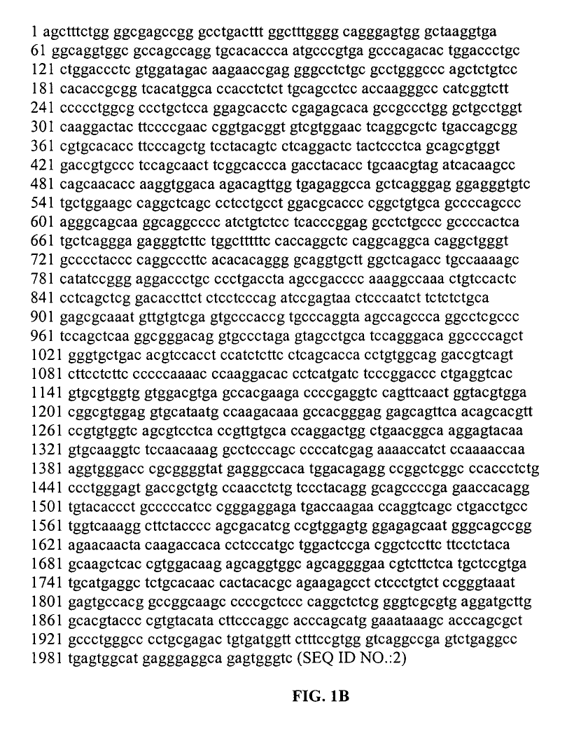

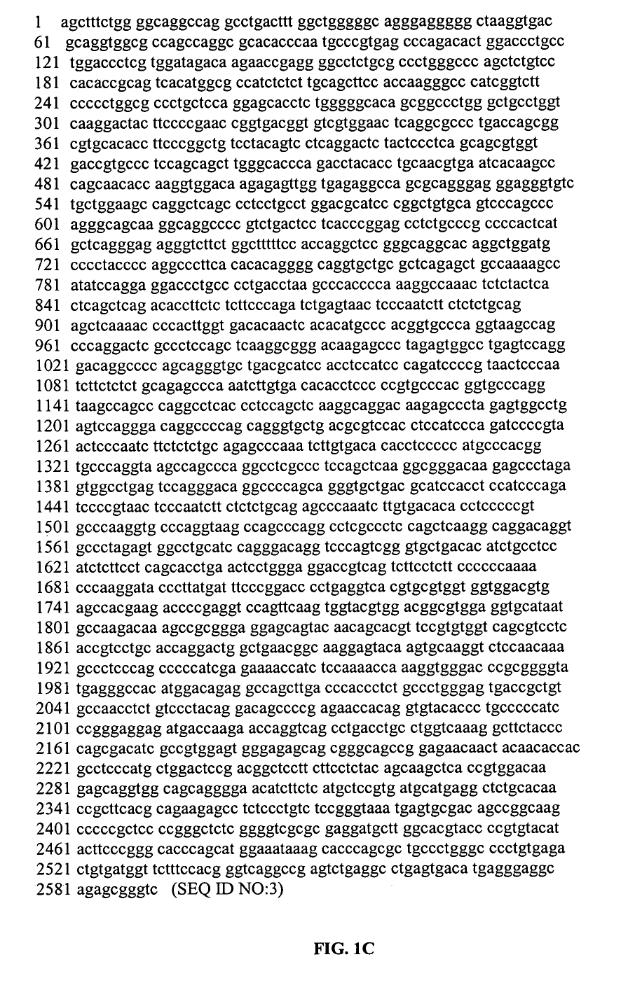

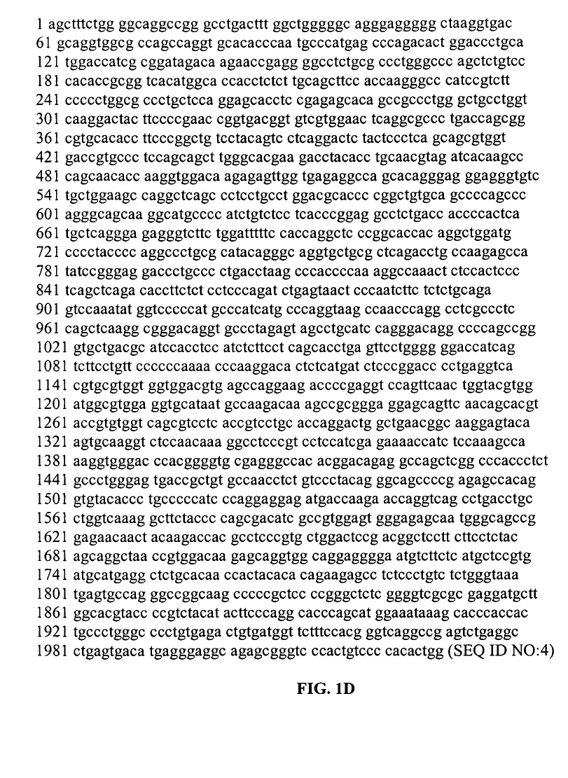

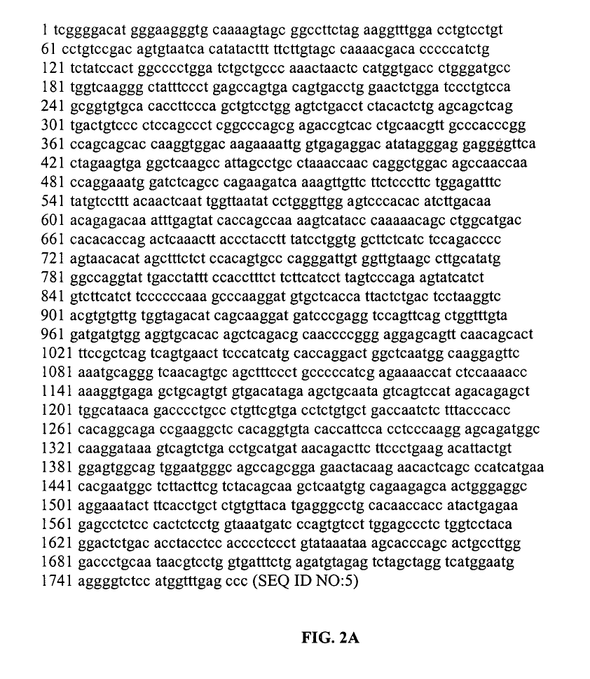

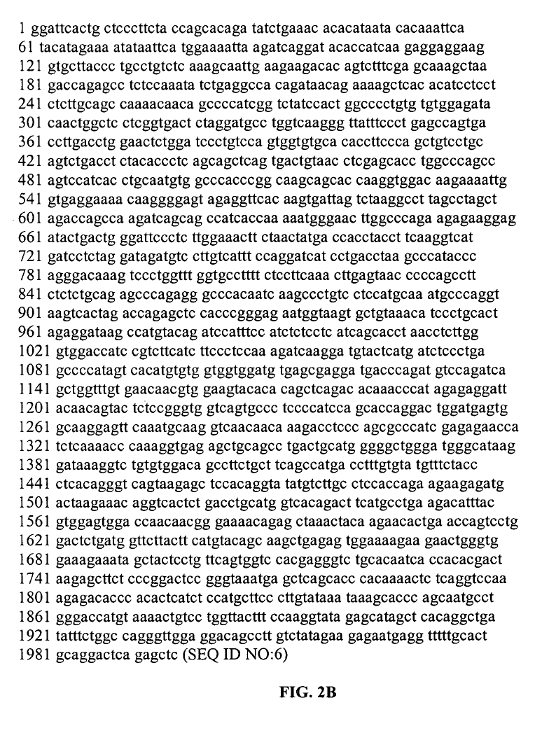

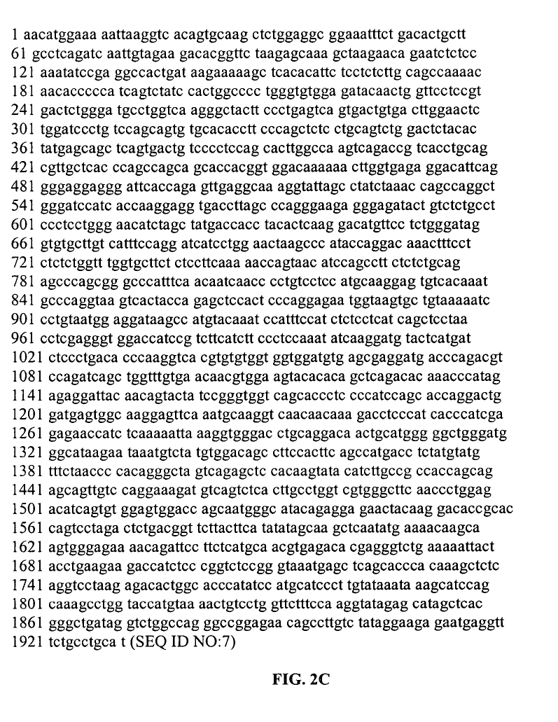

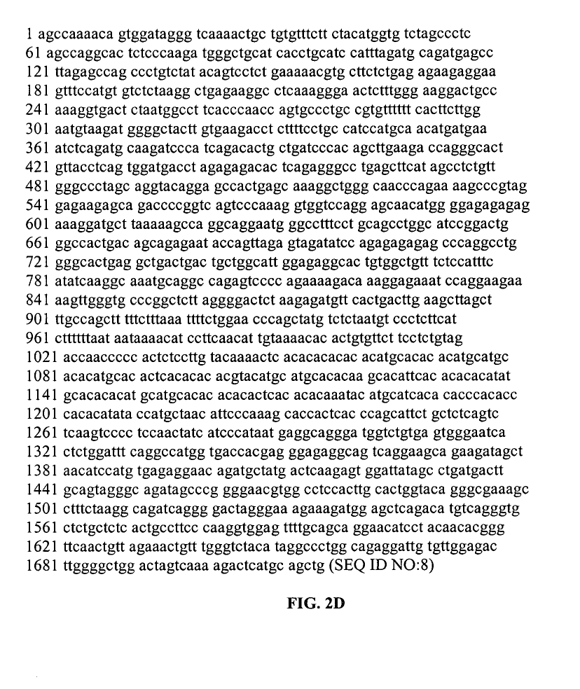

### DETAILED DESCRIPTION OF THE INVENTION

The practice of the present invention will employ, unless otherwise indicated, conventional techniques of cell culture, molecular biology, microbiology, cell biology, and immunology, which are well within the skill of the art. Such techniques are fully explained in the literature. See, e.g., Sambrook et al., 1989, “Molecular Cloning: A Laboratory Manual”, Cold Spring Harbor Laboratory Press; Ausubel et al. (1995), “Short Protocols in Molecular Biology”, “Molecular Cloning: A Laboratory Manual” by T. Maniatis, et al., Cold Spring Harbor Laboratory, Cold Spring Harbor, N.Y., 1982, John Wiley and Sons; Methods in Enzymology (several volumes); Methods in Cell Biology (several volumes), and Methods in Molecular Biology (several volumes).

The present invention encompasses polynucleotides and novel expression vectors which comprise an immunoglobulin gene polyadenylation signal and a DNA sequence encoding a polypeptide wherein the DNA sequence is other than a genomic DNA sequence from the immunoglobulin gene, a eukaryotic cell or cell-line comprising the novel expression vectors and methods of producing a desired polypeptide comprising culturing the mammalian cells transfected with the novel expression vectors and recovering the desired polypeptide from the culture medium. In an additional embodiment, the immunoglobulin gene polyadenylation signal is a polyadenylation signal from an immunoglobulin gene of the IgG isotype. In a further embodiment, the polyadenylation signal is from an IgG heavy-chain gene. In yet another embodiment, the polyadenylation signal is from a human IgG heavy-chain gene. In an additional embodiment, the human IgG heavy-chain gene polyadenylation signal is selected from the group consisting of an IgG1, IgG2, IgG3 and IgG4 heavy-chain gene polyadenylation signal. In a further embodiment, the novel polynucleotide and expression vector comprises a human IgG1 heavy-chain gene polyadenylation signal.

Polynucleotides and Expression Vectors

As used herein, the term “polynucleotide” is a polymer of deoxyribonucleic acid or ribonucleic acid linked together by 5′-3′ phosphodiester bonds between the sugar (deoxyribose or ribose) and phosphate groups. The polynucleotides are composed of repeating nucleotide units and, as disclosed herein, are capable of existing in the double helix configuration wherein the paired guanine/cytosine (G/C) and adenine/thymine (A/T) bases form hydrogen bonds stabilizing the double helix.

As used herein, the term “expression vector” is a vector, such as a DNA vector, that comprises a coding sequence and various regulatory sequences which are necessary for the expression of recombinant polypeptides in cells. One type of expression vector is a plasmid, which refers to a circular double stranded DNA loop into which additional DNA segments may be ligated. The coding sequence may be any suitable coding sequence including a sequence encoding a desired protein, a desired peptide and/or a desired polypeptide. The coding sequence may be derived from any suitable source, for example, a cDNA-derived nucleic acid fragment or a nucleic acid fragment isolated by polymerase chain reaction (PCR).

Regulatory sequences which may be included in an expression vector include a promoter and/or enhancer operably linked to the DNA sequence to be expressed, 5′ or 3′ non-translated sequences such as ribosome binding sites, splice donor and acceptor sites, sequences which control the initiation of transcription (e.g., Kozac sequences, etc.) and sequences which control the termination of transcription and translation, for example, a polyadenylation signal. DNA sequences are operably linked when they are positioned such that they are functionally related to each other. For example, a promoter is operably linked to a DNA coding sequence when it is able to control transcription of the coding sequence. A ribosome binding site is operably linked to a DNA coding sequence when it is positioned so as to assist in translation. A signal peptide is operably linked to a DNA coding sequence when it is expressed as a precursor which participates in the secretion of the polypeptide.

As used herein, an “immunoglobulin gene” is understood to mean a gene from the immunoglobulin gene superfamily. An “immunoglobulin gene polyadenylation signal” is understood to mean a DNA sequence which is a subsequence of the immunoglobulin gene that comprises the AATAAA sequence near the 3′ end of the gene wherein the subsequence is capable of signaling polyadenylation in the transcription of the immunoglobulin gene. Typically, the subsequence from the immunoglobulin gene comprises about 1000 nucleotides. More typically, the immunoglobulin gene polyadenylation signal comprises a subsequence from the immunoglobulin gene of about 700, 600, 500, 400, 300 or fewer nucleotides.

The immunoglobulin gene polyadenylation signal is also a subsequence of an immunoglobulin gene that comprises the AATAAA sequence and a GT/T-rich sequence downstream of the AATAAA sequence wherein the subsequence is capable of signaling polyadenylation in the transcription of the immunoglobulin gene. The GT/T-rich sequence (the GT/T-rich sequence corresponds to the mRNA GU/U rich region) is a sequence of nucleotides found approximately 20-50 nucleotides downstream of the AATAAA sequence and which is a T or GT-rich motif. The immunoglobulin gene polyadenylation signal is also a subsequence of the immunoglobulin gene that comprises the AATAAA sequence, the downstream G/T-rich sequence and an additional downstream GC-rich nucleotide sequence of an immunoglobulin gene wherein the subsequence is capable of signaling polyadenylation in the transcription of the immunoglobulin gene. The GC-rich sequence is a sequence found downstream of the T or G/T-rich sequence and is a GC-rich motif which may form a hairpin loop structure. In addition, the polyadenylation signal is also a subsequence of the immunoglobulin gene that comprises the AATAAA sequence and a sequence found upstream (5′) of the AATAAA sequence in an immunoglobulin gene wherein the subsequence is capable of signaling polyadenylation in the transcription of the immunoglobulin gene.

While the AATAAA sequence described above is preferred, it may be substituted with other hexanucleotide sequences with homology to AATAAA which are capable of signaling polyadenylation in natural mRNAs. These homologous hexanucleotide sequences include ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA and AATAAG. Therefore, in one embodiment, the immunoglobulin gene polyadenylation signal is also a subsequence of an immunoglobulin gene that comprises a hexanucleotide sequence selected from the group consisting of AATAAA, ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, AATAAG and other homologous or substantially identical DNA sequences wherein the subsequence is capable of signaling polyadenylation in the transcription of the immunoglobulin gene. The polyadenylation signal is also a subsequence of an immunoglobulin gene that comprises one of the hexanucleotide sequences described above and a G/T-rich sequence found downstream of the hexanucleotide sequence wherein the subsequence is capable of signaling polyadenylation in the transcription of the immunoglobulin gene. The polyadenylation signal is also a subsequence of an immunoglobulin gene that comprises one of the hexanucleotide sequences described above, the downstream G/T-rich sequence and an additional downstream GC-rich nucleotide sequence wherein the subsequence is capable of signaling polyadenylation in the transcription of the immunoglobulin gene.

The immunoglobulin gene polyadenylation signal may be a subsequence from an immunoglobulin gene of any isotype, including immunoglobulin M (IgM), immunoglobulin D (IgD), immunoglobulin A (IgA1 and IgA2), immunoglobulin E (IgE) and immunoglobulin G (IgG). The polyadenylation signal may be from an immunoglobulin light-chain gene (including λ and κ genes) or from an immunoglobulin heavy-chain gene. In one embodiment of the present invention, the polyadenylation signal is not an immunoglobulin κ light-chain gene polyadenylation signal. The polyadenylation signal may be from any mammalian species. Examples of mammalian species include, but are not limited to, human, primate, bovine, ovine, porcine and rodent species. In one embodiment of the present invention, the immunoglobulin gene polyadenylation signal is not a mouse κ gene polyadenylation signal. In another embodiment, the polyadenylation signal is not a mouse immunoglobulin light-chain gene polyadenylation signal. In a further embodiment, the polyadenylation signal is not a mouse immunoglobulin gene polyadenylation signal.

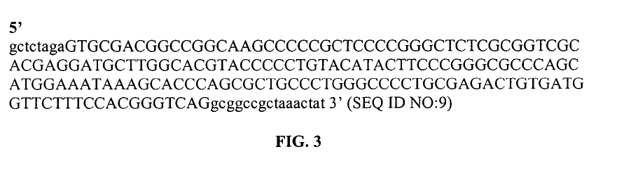

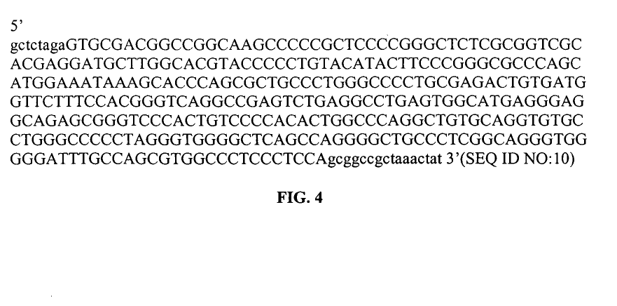

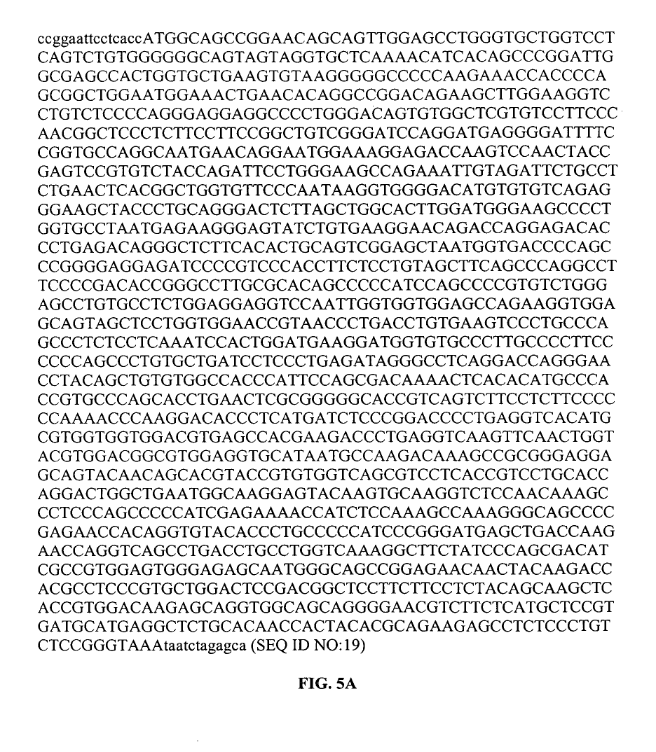

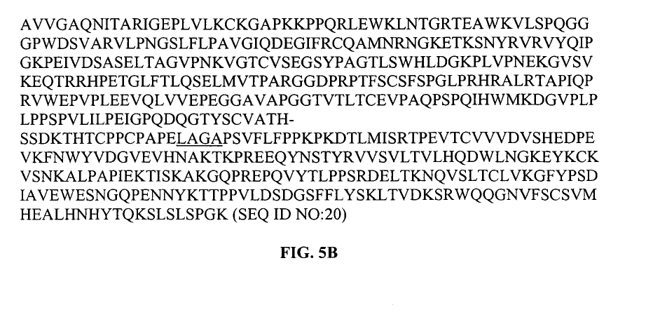

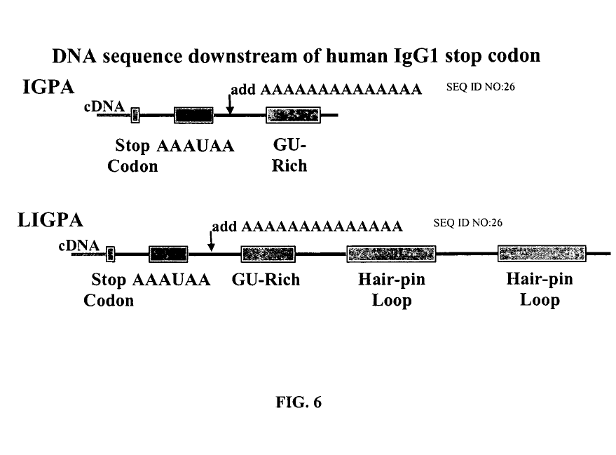

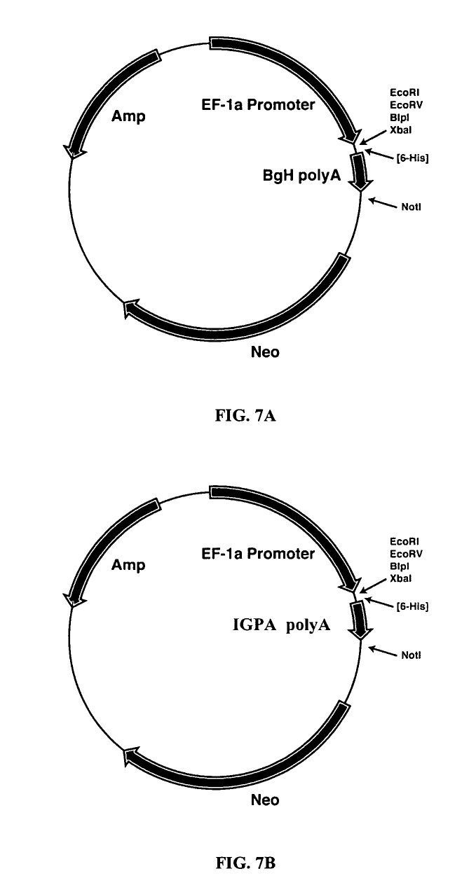

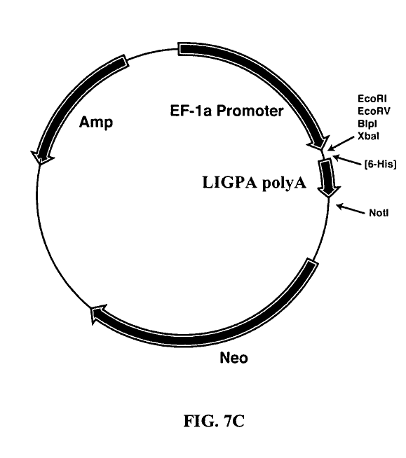

Preferably, the immunoglobulin gene polyadenylation signal is a subsequence from an immunoglobulin gene of an IgG subclass. The IgG gene polyadenylation signal may be selected from any appropriate mammalian species. Polyadenylation signals may be selected from IgG genes including, but not limited to, human IgG subclasses IgG1, IgG2, IgG3 and IgG4 (See Takahashi et al. (1982), Cell, 29(2): 671-9), murine IgG subclasses IgG1, IgG2a, IgG2b and IgG3, rat IgG subclasses IgG1, IgG2a, IgG2b, IgG2c and IgG3, bovine and sheep IgG subclasses IgG1 and IgG2. In one embodiment, the polyadenylation signal is an IgG heavy-chain gene polyadenylation signal. In another embodiment, the polyadenylation signal is selected from the group consisting of a human and murine IgG heavy-chain gene polyadenylation signal. In a further embodiment, the polyadenylation signal is a human or mouse IgG1 heavy-chain gene polyadenylation signal. In an additional embodiment, the polyadenylation signal is a human IgG1 heavy-chain gene polyadenylation signal. In further embodiments, the polyadenylation signal is a nucleotide sequence comprising the sequence of SEQ ID NO:10 (FIG. 4) or a subsequence thereof comprising the sequence of SEQ ID NO:9 (FIG. 3). In other embodiments, the polyadenylation signal is a nucleotide sequence consisting of the sequence of SEQ ID NO: 10 (FIG. 4) or the subsequence thereof, SEQ ID NO:9 (FIG. 3). The polyadenylation signal is additionally a nucleotide sequence which comprises or consists of a nucleotide sequence with homology or sequence identity to SEQ ID NO:9, SEQ ID NO:10 or any of the other polyadenylation signals sequences described herein. As used herein, two sequences have sequence identity or homology when the nucleotide sequences are homologous or identical by at least about 60%, 70%, 80%, 90%, 95% or more. In some embodiments, the sequence identity can be about 90%, 91%, 92%, 93%, 94%, 95%, 96%, 97%, 98%, 99% or higher. Substantial identity also exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

As used herein, the term “hybridizes under low stringency, medium stringency, high stringency, or very high stringency conditions” describes conditions for hybridization and washing. Guidance for performing hybridization reactions can be found in Current Protocols in Molecular Biology (1989) John Wiley & Sons, N.Y., 6.3.1-6.3.6, which is incorporated by reference. Aqueous and nonaqueous methods are described in that reference and either can be used. For applications that require high selectivity, one will typically desire to employ relatively high stringency conditions to form hybrids. In solutions used for some membrane based hybridizations, addition of an organic solvent such as formamide allows the reaction to occur at a lower temperature. High stringency conditions are, for example, relatively low salt and/or high temperature conditions, such as provided by about 0.02 M to about 0.10 M NaCl at temperatures of about 50° C. to about 70° C. Few, if any, mismatches between the probe or primers and the template or target strand would occur under high stringency conditions. In order to achieve less stringent conditions, the salt concentration may be increased and/or the temperature may be decreased. For example, a medium stringency condition could be achieved at a salt concentration of about 0.1 to 0.25 M NaCl at a temperature of about 37° C. to about 55° C., while a low stringency condition could be achieved at a salt concentration of about 0.15 M to about 0.9 M NaCL, at temperatures ranging from about 20° C. to about 55° C. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In another embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a additional embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http:/www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

It is to be understood that the polyadenylation signals of the present invention may include functional equivalents of a polyadenylation signal from an immunoglobulin gene. A functional equivalent is a nucleotide sequence which has been modified, e.g., by substitution of one nucleotide for another, but which is able to signal polyadenylation in substantially the same manner as a polyadenylation signal from an immunoglobulin gene and/or which is able to form substantially the same three-dimensional structure as a polyadenylation signal from an immunoglobulin gene such that the polyadenylation protein complex is able to bind.

The polyadenylation signals described herein may be prepared by procedures well known in the art, including digestion of an immunoglobulin gene nucleotide sequence and/or by chemical synthesis. The DNA sequence comprising the polyadenylation signal can be prepared from an immunoglobulin gene by cleavage with a known restriction endonuclease. Thereafter, the DNA sequence can be used as a polyadenylation signal after cleavage of several additional nucleotides beyond those actually cleaved from the immunoglobulin gene by the restriction endonuclease. The techniques for the use of such restriction endonucleases for accomplishing the cleavage of DNA sequences are well known in the art. Alternatively, additional nucleotides may be added to a polyadenylation signal excised from the immunoglobulin gene to create a larger polyadenylation signal which includes non-genomic sequences. For example, if a genomic immunoglobulin sequence were obtained consisting of 50 nucleotides upstream and 50 nucleotides downstream from the 3′ end, a non-genomic DNA compound might be ligated either upstream or downstream thereof (or both) in order to obtain a substantially larger DNA compound comprising an immunoglobulin gene polyadenylation signal in accordance with the present invention. Finally, total or partial chemical synthesis is optionally employed to prepare any immunoglobulin gene polyadenylation signal described above.

Sequences substantially identical, similar or homologous to an immunoglobulin gene polyadenylation signal disclosed herein are also part of this invention. As discussed above, the polyadenylation signal is a nucleotide sequence comprising the AATAAA sequence or other homologous or substantially identical hexanucleotide sequences from an immunoglobulin gene. The polyadenylation signal is also a nucleotide sequence comprising the AATAAA sequence and a downstream G/T-rich sequence of an immunoglobulin gene. Further, as discussed above, the polyadenylation signal is also a nucleotide sequence comprising the AATAAA sequence, a downstream G/T-rich region and a downstream GC-rich region of an immunoglobulin gene which may form a hairpin loop structure.

In the human IgG1 heavy-chain gene described in GenBank Accession Number J00228, an AATAAA sequence is found at or near nucleotides 1903-1908, a GT/T-rich region is found at or near nucleotide 1941 and a GC-rich region is found at or near nucleotide 1963 to the 3′end of the gene (SEQ ID NO:1). In the human IgG2 heavy-chain gene described in GenBank Accession Number J00230, an AATAAA sequence is found at or near nucleotides 1903-1908, a GT/T-rich region is found at or near nucleotide 1941 and a GC-rich region is found at or about nucleotide 1963 to the 3′ end of the gene (SEQ ID NO:1). In the human IgG3 heavy-chain gene described in GenBank Accession Number X03604, an AATAAA sequence is found at or near nucleotides 2484-2489, a GT/T-rich region is found at or near nucleotide 2514 and a GC-rich region is found at or about nucleotides 2535 to the 3′ end of the gene (SEQ ID NO:3). In the human IgG4 heavy-chain gene described in GenBank Accession Number K01316, an AATAAA sequence is found at or near nucleotides 1904-1909, a GT/T-rich region is found at or near nucleotide 1941 and a GC-rich region is found at or about nucleotide 1964 to the 3′ end (SEQ ID NO:4). In the mouse IgG1 heavy-chain gene described in GenBank Accession Number J00453, an AATAAA sequence is found at or near nucleotides 1716-1721 and a GT/T-rich and GC-rich regions are found downstream of nucleotide 1721 (SEQ ID NO:5). In the mouse IgG2a heavy-chain gene described in GenBank Accession Number J00470, an AATAAA sequence is found at or near nucleotides 1838-1844 and GT/T-rich and GC-rich regions are found downstream of nucleotide 1844 (SEQ ID NO:6). In the mouse IgG2b heavy-chain gene described in GenBank Accession Number J00461, an AATAAA sequence is found at or near nucleotides 1787-1792 and GT/T-rich and GC-rich regions are found downstream of nucleotide 1792 (SEQ ID NO:7). In the mouse IgG3 heavy-chain gene described in GenBank Accession Number X00915, an AATAAA sequence is found at or near nucleotides 6971-6976 and a GT/T-rich and GC-rich regions are found downstream of nucleotide 6976 (SEQ ID NO:8).

In addition to an immunoglobulin gene polyadenylation signal, the novel expression vector comprises a DNA sequence encoding a polypeptide, wherein said DNA sequence is other than a genomic DNA sequence from said immunoglobulin gene. As used herein, a “genomic DNA sequence from an immunoglobulin gene” is a DNA sequence that contains both the exons and the introns from the immunoglobulin gene. The DNA sequence encoding a polypeptide may encode any desired polypeptide with an encoding DNA sequence which is known or can be determined. Such polypeptides include, but are not limited to, proteins such as cytokines (such as TNF-α, IL-1a, IL-1b, IL-4, IL-6, IL-8, IL-10, IL-14, IL-18, IFN-γ, platelet activating factor, macrophage migration inhibitory factor and HMGB1), growth factors (such as granulocyte colony stimulating factor, granulocyte-macrophage colony stimulating factor, mast cell growth factor, stem cell growth factor, epidermal growth factor and growth hormone), protein receptors (such as receptors for cytokines, growth factors, advanced glycation end-products, neurotransmitters and hormones) and high mobility group proteins. In another embodiment, the DNA sequence encoding a polypeptide, encodes for a high mobility group (HMG) protein or a fragment thereof. Preferably, the HMG protein is an HMGB protein or a fragment thereof. More preferably, the HMGB protein is selected from the group consisting of an HMGB1 protein (for example, as described in GenBank Accession Numbers U51677 and U00431), an HMGB2 protein (for example, as described in GenBank Accession Numbers M83665 and X67668) and an HMGB3 protein (for example, as described in GenBank Accession Numbers NM_{—}005342 and Y10044). Fragments of HMGB proteins include, for example, the A and B box fragments (described for example in U.S. Patent Application Publication 2003/0060410 A1). The DNA sequence encoding an HMGB protein or fragment thereof preferably encodes an HMGB 1 protein or fragment thereof.

The DNA sequence encoding a polypeptide may also encode immunoglobulin molecules, or portions thereof, such as antibodies, antibody fragments including Fv, Fab, Fab′ and F(ab′)₂ fragments. Antibodies include chimeric and humanized antibodies or fragments thereof. The DNA sequence encoding a polypeptide may also encode for various fusion proteins including immunoglobulin fusion proteins. DNA sequences encoding such immunoglobulin fusion proteins may be prepared using standard methods. Methods for the preparation of these DNA sequences are described, for example, in U.S. Pat. Nos. 5,116,964, 5,428,130, 5,455,165, 5,514,582, 5,155,027, 5,567,584, 6,018,026, 6,291,646 and 6,323,323.

In addition to a DNA sequence encoding a polypeptide and an immunoglobulin gene polyadenylation signal, the novel expression vector can additionally comprise a suitable promoter which is upstream from and operably linked to the DNA sequence encoding a polypeptide. The promoter utilized in the inventive expression vector can be any promoter which is capable of directing transcription of a DNA sequence encoding a polypeptide when operably linked to said promoter. Commonly utilized promoters which can be used in the inventive expression vectors are derived from Polyoma, Adenovirus, SV40, beta-globin, EF-1a, human cytomegalovirus (CMV), β-actin, α-fetoprotein, γ-globin, β-interferon, Metallothionein II (MT II), amylase, cathepsin, M1 muscarinic receptor, gamma-glutamyl transferase, mouse mammary tumor virus (MMTV) and Rous sarcoma virus (RSV). Suitable CMV promoter sequences can be obtained from the CMV-promoted β-galactosidase expression vector, CMV.beta. (MacGregor et al. (189), Nucl. Acids Res. 17:2365). Suitable amylase promoter, especially murine amylase promoter sequences are described by Wu et al. (Molec. Cell. Biol. 11:4423-4430 (1991)). Suitable cathepsin E promoter sequences are described by Azuma et al. (J. Biol. Chem. 267:1609-1614 (1992)). Suitable M1 muscarinic receptor promoter sequences are described by Fraser et al. (Molec. Pharmacol. 36:840-847 (1989)) and by Bonner (Trends Neurosci. 12:148-151 (1989)). Early and late SV40 promoters are described by Fiers et al. (1978) (Nature 273: 113). Preferably, the polynucleotides and expression vectors comprises a CMV or EF-1a promoter.

Optionally, the novel expression vector further comprises transcription enhancers. Enhancers are cis-acting elements of DNA, usually about 10 to 300 nucleotides in size that act on a promoter to increase its transcription. Many enhancer sequences are now known from mammalian genes including, for example, globin, elastase, albumin, α-fetoprotein, and insulin. Enhancers from a eukaryotic cell virus can also be utilized. Examples include the SV40 enhancer on the late side of the replication origin, the cytomegalovirus early promoter enhancer, the polyoma enhancer on the late side of the replication origin, and adenovirus enhancers. The enhancer can be spliced into the vector at a position 5′ or 3′ to a coding sequence, but is preferably located at a site 5′ from the promoter.

The novel expression vectors can additionally comprise a selectable marker which can be utilized to determine whether the recombinant DNA compound has been incorporated into the expressed genetic material of a living cell. The use of such markers and the principles of selectivity therefor are well known and well recognized in the art. The nucleotide sequence which comprises the selectable marker operates essentially independent of the DNA sequence encoding a desired polypeptide, the promoter and the polyadenylation signal. Accordingly, the location of the selectable marker in the vector relative to the other three sequences is not critical. Selectable markers which can be used in the inventive vectors include antibiotic resistance genes which are useful in determining incorporation into those living cells towards which the antibiotic is otherwise lethal. In mammalian cells, for example, the aminoglycoside antibiotic geneticin (G418) results in inhibition of protein synthesis and death of the cell. Accordingly, the G418 resistance gene comprises a suitable selectable marker. Similarly, the dihydrofolate reductase (DHFR) gene provides a useful marker for cells (e.g., Chinese hamster ovary cells) which can be prepared DHFR deficient.

Optionally, another technique for selection which does not require the utilization of a selectable marker is the use of co-transference techniques whereby both the DNA compound of the present invention absent a marker and a separate DNA molecule containing the marker are co-transferred into a cell. See, for example, U.S. Pat. No. 4,399,216. Incorporation into the expressed genetic material of a higher eukaryotic cell is accomplished by known means. Typically, the incorporation will result in the presence of a DNA compound in the chromosomal structure of the cell, but may also be accomplished by an extra-chromosomal incorporation, e.g., utilization of a recombinant bovine papilloma virus. See Hsiung, et al., J. Molecular and Applied Genetics 2:497-506 (1984) for the methodology for such incorporation.

Eukaryotic Cells Transfected with Novel Expression Vectors and Methods of Producing a Desired Polypeptide

The present invention is also directed to eukaryotic cells or eukaryotic cell-lines transfected with a novel expression vector comprising an immunoglobulin gene polyadenylation signal and a DNA sequence encoding a polypeptide, wherein said DNA sequence is other than a genomic DNA sequence from said immunoglobulin gene and wherein said DNA sequence is upstream from and operably linked to said immunoglobulin gene polyadenylation signal.

One embodiment of the present invention is directed to mammalian cells comprising the inventive, expression vectors described above. Any suitable mammalian cell or cell line may be used in accordance with the present invention. Mammalian cell lines that have been used to express recombinant proteins and which can be used in the present invention include, but are not limited to, COS-7 cells (Guzman et al. (1981), Cell 23: 175), CV-1/EBNA cells (ATCC CRL 10478), L cells, Chinese hamster ovary (CHO) cells, HeLa cells, BHK cells, MDCK cells, 323 cells, 293 cells, MCF-7 cells, NSO cells, SP2 cells and HEK-293 cells. In one embodiment, the mammalian cell comprising the novel expression vector is a CHO cell. The cell or cell-line used to express the desired recombinant protein can also be an immortalized mammalian cell line, which may be of lymphoid origin, such as a myeloma, hybridoma, trioma or quadroma cell line. The cell line can also comprise a normal lymphoid cell, such as a B-cell, which has been immortalized by transformation with a virus, such as the Epstein-Barr virus. The immortalized cell line can, for example, be a myeloma cell line or a derivative thereof. Other suitable host cells are known to those skilled in the art.

A cell or cell-line can be transfected using any suitable method now known or later discovered. Several methods of transfection are known in the art and can be utilized for the practice of the present invention. Known transfection methods include calcium phosphate precipitation, DEAE dextran-mediated gene transfer, liposome-mediated gene transfer, electroporation, microinjection, retroviral transfection and the use of gene guns. The particular transfection method employed will depend on many factors including the eukaryotic cell type utilized and polypeptide to be produced. For example, calcium phosphate precipitation (described for example in Wigler et al. (1980), PNAS 77: 3567) and liposome-mediated gene transfer are useful for the transfection of most mammalian cells but have been found to be more effective with adherent cells. In contrast, electroporation (described for example in Potter et al. (1988), PNAS 81: 7161) is primarily utilized for the transfection of cells in suspension. Retroviral transfection methods are useful for transfection of many mammalian cell types (described for example in Mann et al. (1993), Cell 33:153-159; Pear et al., PNAS USA 90(18):8392-6 (1993); Kitamura et al., PNAS USA 92:9146-9150 (1995)). Various techniques for transfection of mammalian cells are further described in Keown et al., 1990 Methods in Enzymology, 185:527-537, Mansour et al. (1998), Nature, 336:348-352 and Sambrook, et al., Molecular Cloning, 2nd Ed., Vol. 3, Chapter 16, particularly sections 68-72 (1989).

The eukaryotic cell or cell line can be transfected with a novel expression vector comprising a polyadenylation signal from an immunoglobulin gene of any isotype as discussed above. In one embodiment, the polyadenylation signal is from an IgG subclass. In another embodiment, the polyadenylation signal is from an IgG1 gene. In a further embodiment, the polyadenylation signal from an IgG1 heavy-chain gene. In an another embodiment, the polyadenylation signal is from a human IgG1 heavy-chain gene. In another embodiment, a mammalian cell or cell line is transfected with a novel expression vector comprising a human IgG1 heavy-chain gene. In yet another embodiment, the mammalian cell or cell line is a CHO cell or cell line.

The present invention is also directed to a method of producing a desired polypeptide by culturing a mammalian cell transfected with a novel expression vector and recovering said desired polypeptide from the culture medium The polypeptide can be isolated and purified using conventional biochemical methods.

Conditions suitable for growth of the mammalian cell or cell-line and for expression of the vector are well known in the art and are described for example in Sambrook et al., 1989, “Molecular Cloning: A Laboratory Manual.” The desired polypeptide can be recovered after culturing cells for a period of time sufficient to allow expression of the desired polypeptide in the mammalian cell and release of the desired polypeptide into the culture medium where it can be recovered using standard protein purification methods.

The invention is illustrated by the following examples which are not intended to be limiting in any way.

### EXEMPLIFICATION

### Example 1

### Construction of Human IGG1 Heavy-Chain Gene Polyadenylation Signal Sequence 1 (“IGPA”)

A human IgG1 heavy-chain gene polyadenylation signal sequence was assembled by PCR using the overlapping primers designated pIGPA5 and pIGPA3 in Table 1 below.

Briefly, primers were combined in various molar ratios in a cycler reaction of 1 step at 94° C. for 2 minutes, followed by 8 cycles at 94° C. for 2 minutes and 5 minutes at 72° C. with a 30 second ramp time between each step, followed by a step at 72° C. for 10 minutes. The product (“cIGPA”) obtained was then used as a template for the final amplification reaction.

The IGPA sequence was amplified using cIGPA as a template. cIGPA was combined with the overlapping primers designated pIGPA5a and pIGPA3a in Table 1. A standard amplification cycle of 1 step at 94° C. for 2 minutes, followed by 30 cycles at 94° C. for 1 minute, 55° C. for 30 seconds and 72° C. for 5 minutes, followed by a step at 72° C. for 10 minutes, was used. The complete IGPA (shown below) is 187 base pairs in length and is used in the final expression vector and as a template for the 5′ region for IgG1 heavy-chain gene polyadenylation signal sequence 2 (LIGPA). IGPA: SEQ ID NO:9 (lower case indicates cloning sites)

5′ gctctagaGTGCGACGGCCGGCAAGCCCCCGCTCCCCGGGCTCTCGC
GGTCGCACGAGGATGCTTGGCACGTACCCCCTGTACATACTTCCCGGGCG
CCCAGCATGGAAATAAAGCACCCAGCGCTGCCCTGGGCCCCTGCGAGACT
GTGATGGTTCTTTCCACGGGTCAGgcggccgctaaactat 3′

TABLE 1
Primers used to create polyadenylation signal IGPA

| | | |
|---|---|---|
| Primer | Sequence | SEQ ID NO. |

| | | |
|---|---|---|
| pIGPA5 | 5′ CGGCCGGCAAGCCCCCGCTCCCCGGG | SEQ ID NO:10 |
| | CTCTCGCGGTCGCACGAGGATGCTTGGCA | |
| | CGTACCCCCTGTACATACTTCCCGGGCGC | |
| | CCAGC 3′ | |
| pIGPA3 | 5′ AAGAACCATCACAGTCTCGCAGGGGC | SEQ ID NO:11 |
| | CCAGGGCACGGCTGGGTGCTTTATTTCCA | |
| | TGCTGGGCGCCCGGGAAGTATGTAC 3′ | |
| pIGPA5a | 5′ GCTCTAGAGTGCGACGGCCGGCAAGC | SEQ ID NO:12 |
| | CCCCGCTCCCCGGGCTC 3′ | |
| pIGPA3a | 5′ ATAGTTTAGCGGCCGCCTGACCCGTG | SEQ ID NO:13 |
| | GAAAGAACCATCACAGTCTCGCAG 3′ | |

### Example 2

### Construction of Human IGG1 Heavy-Chain Gene Polyadenylation Signal Sequence 2 (“LIGPA”)

A second human IgG1 heavy-chain gene polyadenylation signal sequence was assembled by PCR using the overlapping primers designated as pLIGPA5 and pLIGPA3 in Table 2 below.

Briefly, these primers were combined in various molar ratios in a cycler reaction of 1 step at 94° C. for 2 minutes, followed by 8 cycles at 94° C. for 2 minutes and 72° C. for 5 minutes with a 30 second ramp time between each step, followed by a step at 72° C. for 10 minutes. The product (“cLIGPA3”) obtained was then used as a template for the amplification reaction for the 3′ region of LIGPA.

The 5′ region of the LIGPA sequence (“LIGPA5i”) was amplified using IGPA as a template combined with the overlapping primers designated pIGPA5a in Table 1 and pLIGPA5b in Table 2 below. The 3′ region of LIGPA (“LIGPA3i”) was obtained using cLIGPA3 as a template combined with the overlapping primers designated pLIGPA5a and pLIGPA3a in Table 2 below. Standard amplification cycles of 94° C. for 2 minutes, followed by 30 cycles at 94° C. for 1 minute, 55° C. for 30 seconds and 72° C. for 5 minutes, followed by a step at 72° C. for 10 minutes, were used.

The products of the above two reactions (LIGPA5i and LIGPA3i) were combined in various molar ratios in a cycler reaction of 94° C. for 2 minutes, followed by 8 cycles at 94° C. for 2 minutes and 72° C. for 5 minutes with a 30 second ramping time before each step, followed by a step at 72° C. for 10 minutes. The product of this reaction (“cLIGPA”) served as a template for the final amplification reaction of LIGPA.

The complete LIGPA was amplified using cLIGPA as a template with the primers designated pLIGPA5a and pLIGPA3a in Table 2 below. A standard amplification cycle of one step at 94° C. for 2 minutes, followed by 30 cycles at 94° C. for 1 minute, 55° C. for 30 seconds and 72° C. for 5 minutes, followed by a step at 72° C. for 10 minutes, was used. The product LIGPA (shown directly below) is 347 base pairs in length and is used in the final expression vector.

LIGPA: SEQ ID NO:10 (Lower Case Indicates Cloning Sites)

5′ gctctagaGTGCGACGGCCGGCAAGCCCCCGCTCCCCGGGCTCTCGC
GGTCGCACGAGGATGCTTGGCACGTACCCCCTGTACATACTTCCCGGGCG
CCCAGCATGGAAATAAAGCACCCAGCGCTGCCCTGGGCCCCTGCGAGACT
GTGATGGTTCTTTCCACGGGTCAGGCCGAGTCTGAGGCCTGAGTGGCATG
AGGGAGGCAGAGCGGGTCCCACTGTCCCCACACTGGCCCAGGCTGTGCAG
GTGTGCCTGGGCCCCCTAGGGTGGGGCTCAGCCAGGGGCTGCCCTCGGCA
GGGTGGGGGATTTGCCAGCGTGGCCCTCCCTCCAgcggccgct
aaactat 3′

TABLE 2
Primers used to create polyadenylation signal
LIGPA

| | | |
|---|---|---|
| Primer | Sequence | SEQ ID NO. |

| | | |
|---|---|---|
| pLIGPA5b | 5′ GCCACTCAGGCCTCAGACTCGGCCTG | SEQ ID NO:14 |
| | ACCCGTGGAAAGAACCATCACAGTCTCGC 3′ | |
| pLIGPA5 | 5′ GTGGCATGAGGGAGGCAGAGCGGGTC | SEQ ID NO:15 |
| | CCACTGTCCCCACACTGGCCCAGGCTGTG | |
| | CAGGTGTGCCTGGGCCCCCTAG 3′ | |
| pLIGPA3 | 5′ GGGAGGGCCACGCTGGCAAATCCCCC | SEQ ID NO:16 |
| | ACCCTGCCGAGGGCAGCCCCTGGCTGAGC | |
| | CCCACCCTAGGGGGCCCAGGCACACCTGC | |
| | ACAGC 3′ | |
| pLIGPA5a | 5′ GGCCGAGTCTGAGGCCTGAGTGGCAT | SEQ ID NO:17 |
| | GAGGGAGGCAGAGC 3′ | |
| pLIGPA3a | 5′ ATAGTTTAGCGGCCGCTGGAGGGAGG | SEQ ID NO:18 |
| | GCCACGCTGGCAAATCC 3′ | |

### Example 3

### Construction of Expression Vectors Comprising Either the IgG 1 Heavy-Chain Gene Polyadenylation Signal Sequence 1 (pCTiTOK28) or the IgG1 Heavy-Chain Gene Polyadenylation Signal Sequence 2 (pCTiTOK29)

The mammalian expression vector pcDNA3 (Invitrogen, San Diego, Calif.) was first altered to replace the CMV promoter with the EF-1a promoter for higher protein expression. The pcDNA3 expression vector was also altered to add a Not I site downstream of the BGH polyadenylation signal sequence in order to allow the addition of a second expression cassette. This altered vector is designated pCTiTOK18.

The BGH polyadenylation signal sequence of pCTiTOK18, flanked by Xba I and Not I enzyme restriction sites, was removed and replaced by either the IGPA or the LIGPA polyadenylation signal sequences, which were created with flanking Xba I and Not I enzyme restriction sites. These mammalian expression vectors, which contain either the IGPA or LIGPA polyadenylation signal sequence, are designated pCTiTOK28 and pCTiTOK29, respectively.

The vectors pCTiTOK18, pCTiTOK28 and pCTiTOK29 all contain the gene for ampicillin selection in bacteria and geneticin (G418) selection in mammalian cells. The promoter, EF-1a, is separated from the polyadenylation signal sequences by a region of multiple cloning sites into which a desired cDNA may be functionally cloned.

### Example 4

### RAGE-IG Fusion Protein Production

The RAGE-Ig fusion protein was created by PCR assembly in which two overlapping cDNA fragments were created, annealed by PCR and used as a template for the final PCR product. The extracellular region of RAGE (e.g., amino acids 1-305 of GenBank Accesssion No. NP_{—}001127; SEQ ID NO:19) along with its signal sequence was amplified using human splenic cDNA as a template and the primers designated pTOK16a5 and pTOK17aA, described in Table 3 below. The primer pTOK16a5 contains the sequence for an EcoRI restriction site and a Kozak sequence. The primer pTOK17aA contains 35 bases that overlap with the 5′ region of the human IgG1 Fc.

The human IgG1 Fc region was amplified from a previous human IgG1 fusion protein in which the Fc receptor region was removed by mutation (L235A and G237A). The sequence used covered the hinge CH2 and CH3 regions (e.g., amino acids 219-447 of GenBank Accession No. J00228) and was amplified using the primers pTOK17aB and pTOK10a3. The primer pTOK17aB contained 35 bases that overlapped with the 3′ region of the extracellular region of RAGE and the primer pTOK10a3 contains a stop codon and the sequence for the XbaI restriction site. The PCR cycle involved 1 step at 94° C. for 2 minutes, followed by 30 cycles at 94° C. for 1 minute, 55° C. for 30 seconds and 72° C. for 5 minutes, followed by a step of 72° C. for 10 minutes. The two fragments were then gel purified and combined in equal molar ratios in a PCR reaction without primers that used a PCR cycle involving one step at 94° C. for 2 minutes, followed by 8 cycles of 94° C. for 2 minutes and 72° C. for 5 minutes with a 30-second ramping time at each step, followed by a step at 72° C. for 10 minutes. The product of this reaction then served as a template in another amplification reaction using the primers pTOK16a5 and pTOK10a3 with the same cycler steps used in the first amplification. This product was TA-cloned and sequenced to find the correct cDNA construct. The product, a RAGE-Ig fusion polypeptide, is described as SEQ ID NO:20.

The three expression vectors pCTiTOK17 (BGHpA), pCTiTOK30 (IGPA) and pCTiTOK31 (LIGPA) containing the RAGE-Ig fusion cDNA were all created by functionally cloning the EcoRI-XbaI fragment containing the RAGE-Ig fusion cDNA into the vectors pCTiTOK18, pCTiTOK28 and pCTiTOK29 respectively. After the sequence confirmed, DNA was maxiprepped and transfected into CHO cells as described below.

TABLE 3
Primers used in the creation of the RAGE-Ig fusion
construct

| | | |
|---|---|---|
| Primer | Sequence | SEQ ID NO. |

| | | |
|---|---|---|
| pTOK16a5 | 5′ CCGGAATTCCTCACCATGGCAGCCGG | SEQ ID NO:21 |
| | AACAGCAGTTGGAG 3′ | |
| pTOK17aA | 5′GTTTTGTCGCTGGAATGGGTGGCCAC | SEQ ID NO:22 |
| | ACAGCTGTAGGTTCCCTGGTCCTGAG 3′ | |
| pTOK17aB | 5′GCTGTGTGGCCACCCATTCCAGCGACA | SEQ ID NO:23 |
| | AAACTCACACATGCCCACCGTGCCCAGCA | |
| | CCTG 3′ | |
| pTOK10a3 | 5′ TGCTCTAGATTATTTACCCGGAGACA | SEQ ID NO:24 |
| | GGGAGAGGCCTTCTGCGTGTAGTGGTTGT | |
| | GCAGAGCCTCATGCATCACGG 3′ | |

### Example 5

### Preparation of CHO Cell Transfectants

This example describes the transfection of CHO cells with the constructs pCTiTOK18, pCTiTOK28 and pCTiTOK29. CHO—S cells (Gibco/Invitrogen, Carlsbad, Calif.) were transfected according to the manufacturer's instructions using recommended reagents. Briefly, different concetrations of either of the expression vectors pCTiTOK18, pCTiTOK28 or pCTiTOK29 and the DMRIE-C reagent, a mixture of charged and neutral lipids, were mixed to form complexes for 30-45 minutes. These complexes were then mixed with the CHO cells in serum-free DMEM in the absence of antibiotics. The cells take up the complexes and incorporate the DNA into their chromosomes over the next 5 hours. The transfection was stopped by the addition of media containing 10% fetal bovine serum. The CHO cells were then allowed to recover and incubated at 37° C. overnight in 8% CO₂.

The successfully transfected cells were selected for growth in media containing 10% fetal bovine serum and 50 mg/ml geneticin for 5 days. Single cell clones were then plated into 96-well plates, isolated for the production of human IgG Fc containing protein and selected for an additional 14 days. Single cell clones were then identified and their supernatants were tested for the presence of recombinant protein. Clones determined to be producing the recombinant protein were expanded and adapted to protein-free media (the clones may also be kept in 10% fetal bovine serum serum, however, the presence of serum increases the difficulty of purification). The CHO—S cells easily adapt to growth in Gibco/Invitrogen's protein-free CD-CHO media and shaker flask culture which allows for rapid expansion on the cells. Cell culture media was then collected and the presence of RAGE-Ig fusion protein was determined as described below.

### Example 6

### RAGE-IG Fusion Protein Production by Cells Transfected with pCTiTOK17 (BGHpA), pCTiTOK30 (IGPA) and pCTiTOK31 (LIGPA)

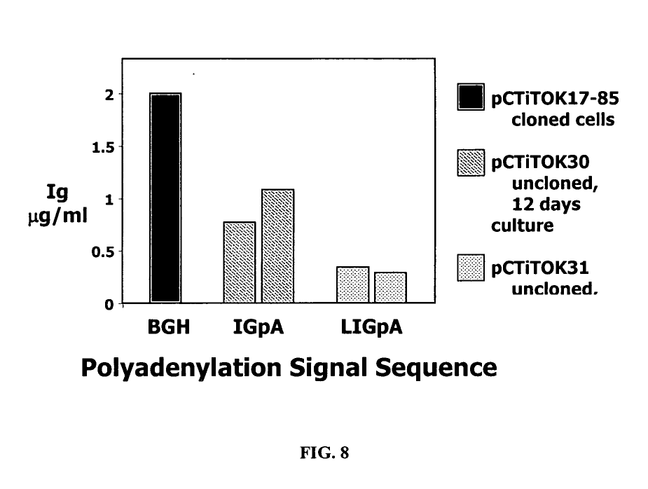

The presence and quantity of RAGE-Ig fusion protein in the cell culture media of CHO cells transfected with either the expression vector pCTiTOK17, pCTiTOK30 or pCTiTOK31, was determined using a standardized ELISA. Briefly, ELISA plates were coated with 0.4 ug/ml of goat anti-human IgG (Jackson ImmunoResearch Laboratories, West Grove, Pa.) and incubated overnight at 4° C. The plates were then blocked with PBS containing 1% BSA for one hour at room temperature. After washing, the plates were incubated with the CHO cells supernatants obtained as described above. After 2 hours at room temperature, the plates were washed and incubated with mouse anti-human IgG-HRP (Jackson ImmunoResearch Laboratories, West Grove, Pa.) diluted at 1:2000 in PBS. After washing, the plates were developed with TMB (Invitrogen, San Diego, Calif.) and absorbance at 655 nm was measured using a plate reader. The amount of RAGE-Ig fusion protein was quantified by comparison to a standard dilution curve of human IgG (Jackson ImmunoResearch Laboratories, West Grove, Pa.). The results of this quantification is presented in FIG. 8. The protein may also be quantified using Western analysis and functional assays.

FIG. 8 shows the results of a preliminary experiment measuring the quantity of RAGE-Ig fusion protein (ug/ml) found in the supernatants of CHO cells transfected with pCTiTOK17, pCTiTOK30 or pCTiTOK31 (comprising the BGH, IGPA or LIGPA polyadenylation signal sequence, respectively). CHO cells transfected with pCTiTOK17 were cloned cells; consequently it was estimated that 100% of the cells were able to produce the RAGE-Ig fusion protein. The cells transfected with pCTiTOK30 and pCTiTOK31 were uncloned cells (i.e., primary pools of transfectants) cultured for 12 and 6 days, respectively. Because the latter cells are uncloned, it is estimated that less than 10% of the cells are able to produce the RAGE-Ig fusion protein. The two bars for the IGPA and LIGPA containing cells represent the results of two separate transfections. A greater quantity of recombinant protein in the supemantants indicates that the protein is properly folded and secreted from the cells, which is suggestive of proper polyadenylation. As shown in FIG. 8, the data suggest that cells transfected with an expression vector comprising either the IGPA or LIGPA polyadenylation signal sequence are able to produce the RAGE-Ig fusion protein

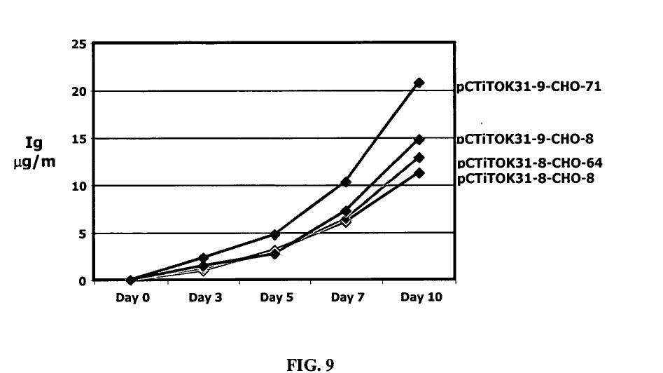

FIG. 9 shows the results of an experiment measuring the quantity of RAGE-Ig fusion protein (ug/ml) in the supernatants from four CHO cell clones transfected with the vector pCTiTOK31 (comprising LIGPA) over a 10 day period. On Day 0, the starting number of cells for each of the five clones was 1.3×10⁸ cells in 750 ml CD-CHO/G418. As shown in FIG. 9, the quantity of RAGE-Ig fusion protein produced by the cells increases over time and by the tenth day, the cells produce a quantity of RAGE-Ig fusion protein ranging from about 11 to 21 ug/ml.

The teachings of all publications cited herein are incorporated herein by reference in their entirety.

While this invention has been particularly shown and described with references to the preferred embodiments thereof, it will be understood by those skilled in the art that various changes in form and details may be made therein without departing from the scope of the invention encompassed by the appended claims.

1. A polynucleotide comprising:
a) an IgG heavy-chain gene polyadenylation signal, and
b) a DNA sequence encoding a polypeptide,
wherein said DNA sequence is other than a genomic DNA sequence from said IgG heavy-chain gene, and wherein said DNA sequence is upstream from and operably linked to said IgG heavy-chain gene polyadenylation signal.

2. The polynucleotide of claim 1, wherein said IgG heavy-chain gene polyadenylation signal is a human IgG heavy-chain gene polyadenylation signal.

3. The polynucleotide of claim 2, wherein said human IgG heavy chain gene polyadenylation signal is selected from the group consisting of an IgG1 heavy-chain gene polyadenylation signal, an IgG2 heavy-chain gene polyadenylation signal, an IgG3 heavy-chain gene polyadenylation signal and an IgG4 heavy-chain gene polyadenylation signal.

4. The polynucleotide of claim 3, wherein said human IgG heavy-chain gene polyadenylation signal is an IgG1 heavy-chain gene polyadenylation signal.

5. The polynucleotide of claim 4, wherein said IgG1 heavy chain gene polyadenylation signal comprises the nucleotide sequence of SEQ ID NO:9.

6. The polynucleotide of claim 5, wherein said IgG1 heavy chain gene polyadenylation signal comprises the nucleotide sequence of SEQ ID NO:10.

7. The polynucleotide of claim 1, further comprising a promoter sequence upstream from and operably linked to said DNA sequence encoding a polypeptide.

8. The expression vector of claim 7, wherein said promoter is selected from the group consisting of an EF-1a promoter and a CMV promoter.

9. The expression vector of claim 8, further comprising a selectable marker.

10. The polynucleotide of claim 4, wherein said DNA sequence encodes an immunoglobulin fusion protein.

11. The polynucleotide of claim 4, wherein said DNA sequence encodes a high mobility group protein.

12. The polynucleotide of claim 11, wherein said high mobility group protein is a HMGB protein.

13. The polynucleotide of claim 12, wherein said HMGB protein is selected from the group consisting of HMGB1, HMGB2 and HMGB3.

14. An expression vector comprising:
a) an IgG heavy-chain gene polyadenylation signal, and
b) a DNA sequence encoding a polypeptide,
wherein said DNA sequence is other than a genomic DNA sequence from said IgG heavy-chain gene, and wherein said DNA sequence is upstream from and operably linked to said IgG heavy-chain gene polyadenylation signal.

15. The expression vector of claim 14, wherein said IgG heavy-chain gene polyadenylation signal is a human IgG heavy-chain gene polyadenylation signal.

16. The expression vector of claim 15, wherein said human IgG heavy chain gene polyadenylation signal is selected from the group consisting of an IgG1 heavy-chain gene polyadenylation signal, an IgG2 heavy-chain gene polyadenylation signal, an IgG3 heavy-chain gene polyadenylation signal and an IgG4 heavy-chain gene polyadenylation signal.

17. The expression vector of claim 16, wherein said human IgG heavy-chain gene polyadenylation signal is an IgG1 heavy-chain gene polyadenylation signal.

18. The expression vector of claim 17, wherein said IgG1 heavy chain gene polyadenylation signal comprises the nucleotide sequence of SEQ ID NO:9.

19. The expression vector of claim 17, wherein said IgG1 heavy chain gene polyadenylation signal comprises the nucleotide sequence of SEQ ID NO:10.

20-28. (canceled)

29. A method of producing a desired polypeptide, said method comprising:
a) culturing a mammalian cell transfected with an expression vector, wherein said vector comprises:
i) an IgG heavy-chain gene polyadenylation signal, and
ii) a DNA sequence encoding a desired polypeptide,
wherein said DNA sequence is other than a genomic DNA sequence from said IgG heavy-chain gene, and wherein said DNA sequence is upstream from and operably linked to said IgG heavy-chain gene polyadenylation signal; and
b) recovering said desired polypeptide.

30. The method of claim 29, wherein said IgG heavy-chain gene polyadenylation signal is a human IgG heavy-chain gene polyadenylation signal.

31. The method of claim 30, wherein said human IgG heavy chain gene polyadenylation signal is selected from the group consisting of an IgG1 heavy-chain gene polyadenylation signal, an IgG2 heavy-chain gene polyadenylation signal, an IgG3 heavy-chain gene polyadenylation signal and an IgG4 heavy-chain gene polyadenylation signal.

32. The method of claim 31, wherein said human IgG heavy-chain gene polyadenylation signal is an IgG1 heavy-chain gene polyadenylation signal.

33. The method of claim 32, wherein said IgG1 heavy chain gene polyadenylation signal comprises the nucleotide sequence of SEQ ID NO:9.

34. The method of claim 32, wherein said IgG1 heavy chain gene polyadenylation signal comprises the nucleotide sequence of SEQ ID NO: 10.

35-46. (canceled)


SEQUENCE LISTING (26 sequences)

SEQ ID NO: 1 - DNA, 2009 bp, Homo sapiens
agctttctggggcaggccaggcctgaccttggctttggggcagggagggggctaaggtgaggcaggtggcgccagcaggtgcacacccaatgcccatgagcccagacactggacgctgaacctcgcggacagttaagaacccaggggcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtc

SEQ ID NO: 2 - DNA, 2009 bp, Homo sapiens
agctttctggggcgagccgggcctgactttggctttggggcagggagtgggctaaggtgaggcaggtggcgccagccaggtgcacacccaatgcccgtgagcccagacactggaccctgcctggaccctcgtggatagacaagaaccgaggggcctctgcgcctgggcccagctctgtcccacaccgcggtcacatggcaccacctctcttgcagcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggtgagaggccagctcagggagggagggtgtctgctggaagccaggctcagccctcctgcctggacgcaccccggctgtgcagccccagcccagggcagcaaggcaggccccatctgtctcctcacccggaggcctctgcccgccccactcatgctcagggagagggtcttctggctttttccaccaggctccaggcaggcacaggctgggtgcccctaccccaggcccttcacacacaggggcaggtgcttggctcagacctgccaaaagccatatccgggaggaccctgcccctgacctaagccgaccccaaaggccaaactgtccactccctcagctcggacaccttctctcctcccagatccgagtaactcccaatcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagaggccggctcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccacggccggcaagcccccgctccccaggctctcggggtcgcgtgaggatgcttggcacgtaccccgtgtacatacttcccaggcacccagcatggaaataaagcacccagcgctgccctgggcccctgcgagactgtgatggttctttccgtgggtcaggccgagtctgaggcctgagtggcatgagggaggcagagtgggtc

SEQ ID NO: 3 - DNA, 2590 bp, Homo sapiens
agctttctggggcaggccaggcctgactttggctgggggcagggagggggctaaggtgacgcaggtggcgccagccaggcgcacacccaatgcccgtgagcccagacactggaccctgcctggaccctcgtggatagacaagaaccgaggggcctctgcgccctgggcccagctctgtcccacaccgcagtcacatggcgccatctctcttgcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttggtgagaggccagcgcagggagggagggtgtctgctggaagccaggctcagccctcctgcctggacgcatcccggctgtgcagtcccagcccagggcagcaaggcaggccccgtctgactcctcacccggagcctctgcccgccccactcatgctcagggagagggtcttctggctttttccaccaggctccgggcaggcacaggctggatgcccctaccccaggcccttcacacacaggggcaggtgctgcgctcagagctgccaaaagccatatccaggaggaccctgcccctgacctaagcccaccccaaaggccaaactctctactcactcagctcagacaccttctctcttcccagatctgagtaactcccaatcttctctctgcagagctcaaaaccccacttggtgacacaactcacacatgcccacggtgcccaggtaagccagcccaggactcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccacggtgcccaggtaagccagcccaggcctcaccctccagctcaaggcaggacaagagccctagagtggcctgagtccagggacaggccccagcagggtgctgacgcgtccacctccatcccagatccccgtaactcccaatcttctctctgcagagcccaaatcttgtgacacacctcccccatgcccacggtgcccaggtaagccagcccaggcctcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccaaggtgcccaggtaagccagcccaggcctcgccctccagctcaaggcaggacaggtgccctagagtggcctgcatccagggacaggtcccagtcgggtgctgacacatctgcctccatctcttcctcagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggtgggacccgcggggtatgagggccacatggacagaggccagcttgacccaccctctgccctgggagtgaccgctgtgccaacctctgtccctacaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacagccggcaagcccccgctccccgggctctcggggtcgcgcgaggatgcttggcacgtaccccgtgtacatacttcccgggcacccagcatggaaataaagcacccagcgctgccctgggcccctgtgagactgtgatggttctttccacgggtcaggccgagtctgaggcctgagtgacatgagggaggcagagcgggtc

SEQ ID NO: 4 - DNA, 2028 bp, Homo sapiens
agctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctcccggcaccacaggctggatgcccctaccccaggccctgcgcatacagggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacacggacagaggccagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatgagtgccagggccggcaagcccccgctccccgggctctcggggtcgcgcgaggatgcttggcacgtaccccgtctacatacttcccaggcacccagcatggaaataaagcacccaccactgccctgggcccctgtgagactgtgatggttctttccacgggtcaggccgagtctgaggcctgagtgacatgagggaggcagagcgggtcccactgtccccacactgg

SEQ ID NO: 5 - DNA, 1763 bp, Mus musculus
tcggggacatgggaagggtgcaaaagtagcggccttctagaaggtttggacctgtcctgtcctgtccgacagtgtaatcacatatactttttcttgtagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctggagtctgacctctacactctgagcagctcagtgactgtcccctccagccctcggcccagcgagaccgtcacctgcaacgttgcccacccggccagcagcaccaaggtggacaagaaaattggtgagaggacatatagggaggaggggttcactagaagtgaggctcaagccattagcctgcctaaaccaaccaggctggacagccaaccaaccaggaaatggatctcagcccagaagatcaaaagttgttcttctcccttctggagatttctatgtcctttacaactcaattggttaatatcctgggttggagtcccacacatcttgacaaacagagacaaatttgagtatcaccagccaaaagtcatacccaaaaacagcctggcatgaccacacaccagactcaaacttaccctacctttatcctggtggcttctcatctccagaccccagtaacacatagctttctctccacagtgcccagggattgtggttgtaagccttgcatatgggccaggtattgacctatttccacctttcttcttcatccttagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggtgagagctgcagtgtgtgacatagaagctgcaatagtcagtccatagacagagcttggcataacagacccctgccctgttcgtgacctctgtgctgaccaatctctttacccacccacaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgatcccagtgtccttggagccctctggtcctacaggactctgacacctacctccacccctccctgtataaataaagcacccagcactgccttgggaccctgcaataacgtcctggtgatttctgagatgtagagtctagctaggtcatggaatgaggggtctccatggtttgagccc

SEQ ID NO: 6 - DNA, 1996 bp, Mus musculus
ggattcactgctcccttctaccagcacagatatctgaaacacacataatacacaaattcatacatagaaaatataattcatggaaaattaagatcaggatacaccatcaagaggaggaaggtgcttaccctgcctgtctcaaagcaattgaagaagacacagtctttcgagcaaagctaagaccagagcctctccaaatatctgaggccacagataacagaaaagctcacacatcctcctctcttgcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattggtgaggaaaacaaggggagtagaggttcacaagtgattagtctaaggccttagcctagctagaccagccaagatcagcagccatcaccaaaaatgggaacttggcccagaagagaaggagatactgactgggattccctcttggaaacttctaactatgaccacctaccttcaaggtcatgatcctctaggatagatgtccttgtcatttccaggatcatcctgacctaagcccatacccagggacaaagtccctggtttggtgccttttctccttcaaacttgagtaacccccagccttctctctgcagagcccagagggcccacaatcaagccctgtcctccatgcaaatgcccaggtaagtcactagaccagagctccacccgggagaatggtaagtgctgtaaacatccctgcactagaggataagccatgtacagatccatttccatctctcctcatcagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtgtatgtttctaccctcacagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatgagctcagcacccacaaaactctcaggtccaaagagacacccacactcatctccatgcttcccttgtataaataaagcacccagcaatgcctgggaccatgtaaaactgtcctggttactttccaaggtatagagcatagctcacaggctgatatttctggccagggttggaggacagccttgtctatagaagagaatgaggtttttgcactgcaggactcagagctc

SEQ ID NO: 7 - DNA, 1931 bp, Mus musculus
aacatggaaaaattaaggtcacagtgcaagctctggaggcggaaatttctgacactgcttgcctcagatcaattgtagaagacacggttctaagagcaaagctaagaacagaatctctccaaatatccgaggccactgataagaaaaagctcacacattctcctctcttgcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacttggccaagtcagaccgtcacctgcagcgttgctcacccagccagcagcaccacggtggacaaaaaacttggtgagaggacattcaggggaggagggattcaccagagttgaggcaaaggtattagcctatctaaaccagccaggctgggatccatcaccaaggaggtgaccttagcccagggaagagggagatactgtctctgcctccctcctgggaacatctagctatgaccacctacactcaaggacatgttcctctgggataggtgtgcttgtcatttccaggatcatcctggaactaagcccataccaggacaaactttcctctctctggtttggtgcttctctccttcaaaaaccagtaacatccagccttctctctgcagagcccagcgggcccatttcaacaatcaacccctgtcctccatgcaaggagtgtcacaaatgcccaggtaagtcactaccagagctccactcccaggagaatggtaagtgctgtaaaaatccctgtaatggaggataagccatgtacaaatccatttccatctctcctcatcagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaaggtgggacctgcaggacaactgcatgggggctgggatgggcataagaataaatgtctatgtggacagccttccacttcagccatgacctctatgtatgtttctaaccccacagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctagactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatgagctcagcacccacaaagctctcaggtcctaagagacactggcacccatatccatgcatcccttgtataaataaagcatccagcaaagcctggtaccatgtaaaactgtcctggttctttccaaggtatagagcatagctcacgggctgataggtctggccagggccggagaacagccttgtctataggaagagaatgaggtttctgcctgcat

SEQ ID NO: 8 - DNA, 1715 bp, Mus musculus
agccaaaacagtggatagggtcaaaactgctgtgtttcttctacatggtgtctagccctcagccaggcactctcccaagatgggctgcatcacctgcatccatttagatgcagatgagccttagagccagccctgtctatacagtcctctgaaaaacgtgcttctctgagagaagaggaagtttccatgtgtctctaaggctgagaaggcctcaaagggaactctttgggaaggactgccaaaggtgactctaatggccttcacccaaccagtgccctgccgtgttttttcacttcttggaatgtaagatggggctacttgtgaagacctcttttcctgccatccatgcaacatgatgaaatctcagatgcaagatcccatcagacactgctgatcccacagcttgaagaccagggcactgttacctcagtggatgacctagagagacactcagagggcctgagcttcatagcctctgttgggccctagcaggtacaggagccactgagcaaaggctgggcaacccagaaaagcccgtaggagaagagcagaccccggtcagtcccaaaggtggtccaggagcaacatggggagagagagaaaggatgcttaaaaagccaggcaggaatgggcctttcctgcagcctggcatccggactgggccactgacagcagagaataccagttagagtagatatccagagagagagcccaggcctggggcactgaggctgactgactgctggcattggagaggcactgtggctgtttctccatttcatatcaaggcaaatgcaggccagagtccccagaaaagacaaaggagaaatccaggaagaaaagttgggtgcccggctcttaggggactctaagagatgttcactgacttgaagcttagctttgccagctttttctttaaattttctggaacccagctatgtctctaatgtccctcttcatcttttttaataataaaacatccttcaacattgtaaaacacactgtgttcttcctctgtagaccaacccccactctccttgtacaaaactcacacacacacacatgcacacacatgcatgcacacatgcacactcacacacacgtacatgcatgcacacaagcacattcacacacacatatgcacacacatgcatgcacacacacactcacacacaaatacatgcatcacacacccacacccacacatataccatgctaacattcccaaagcaccactcacccagcattctgctctcagtctcaagtcccctccaactatcatcccataatgaggcagggatggtctgtgagtgggaatcactctggatttcaggccatggtgaccacgagggagaggcagtcaggaagcagaagatagctaacatccatgtgagaggaacagatgctatgactcaagagtggattatagcctgatgacttgcagtagggcagatagcccggggaacgtggcctccacttgcactggtacagggcgaaagcctttctaaggcagatcaggggactagggaaagaaagatggagctcagacatgtcagggtgctctgctctcactgccttcccaaggtggagttttgcagcaggaacatcctacaacacgggttcaactgttagaaactgtttgggtctacataggccctggcagaggattgtgttggagacttggggctggactagtcaaaagactcatgcagctg

SEQ ID NO: 9 - DNA, 187 bp, Artificial Sequence
  IGPA polyadenylation signal sequence
gctctagagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggcggccgctaaactat

SEQ ID NO: 10 - DNA, 347 bp, Artificial Sequence
  LIGPA polyadenylation signal sequence
gctctagagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccccctagggtggggctcagccaggggctgccctcggcagggtgggggatttgccagcgtggccctccctccagcggccgctaaactat

SEQ ID NO: 11 - DNA, 80 bp, Artificial Sequence
  Oligonucleotide primer
aagaaccatcacagtctcgcaggggcccagggcacggctgggtgctttatttccatgctgggcgcccgggaagtatgtac

SEQ ID NO: 12 - DNA, 43 bp, Artificial Sequence
  Oligonucleotide primer
gctctagagtgcgacggccggcaagcccccgctccccgggctc

SEQ ID NO: 13 - DNA, 50 bp, Artificial Sequence
  Oligonucleotide primer
atagtttagcggccgcctgacccgtggaaagaaccatcacagtctcgcag

SEQ ID NO: 14 - DNA, 55 bp, Artificial Sequence
  Oligonucleotide primer
gccactcaggcctcagactcggcctgacccgtggaaagaaccatcacagtctcgc

SEQ ID NO: 15 - DNA, 77 bp, Artificial Sequence
  Oligonucleotide primer
gtggcatgagggaggcagagcgggtcccactgtccccacactggcccaggctgtgcaggtgtgcctgggccccctag

SEQ ID NO: 16 - DNA, 89 bp, Artificial Sequence
  Oligonucleotide primer
gggagggccacgctggcaaatcccccaccctgccgagggcagcccctggctgagccccaccctagggggcccaggcacacctgcacagc

SEQ ID NO: 17 - DNA, 40 bp, Artificial Sequence
  Oligonucleotide primer
ggccgagtctgaggcctgagtggcatgagggaggcagagc

SEQ ID NO: 18 - DNA, 43 bp, Artificial Sequence
  Oligonucleotide primer
atagtttagcggccgctggagggagggccacgctggcaaatcc

SEQ ID NO: 19 - DNA, 1629 bp, Artificial Sequence
  RAGE-Ig fusion protein
ccggaattcctcaccatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagcgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagagca

SEQ ID NO: 20 - protein, 516 aa, Artificial Sequence
  RAGE-Ig fusion protein
AVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 40 bp, Artificial Sequence
  Oligonucleotide primer
ccggaattcctcaccatggcagccggaacagcagttggag

SEQ ID NO: 22 - DNA, 52 bp, Artificial Sequence
  Oligonucleotide primer
gttttgtcgctggaatgggtggccacacagctgtaggttccctggtcctgag

SEQ ID NO: 23 - DNA, 60 bp, Artificial Sequence
  Oligonucleotide primer
gctgtgtggccacccattccagcgacaaaactcacacatgcccaccgtgcccagcacctg

SEQ ID NO: 24 - DNA, 76 bp, Artificial Sequence
  Oligonucleotide primer
tgctctagattatttacccggagacagggagaggccttctgcgtgtagtggttgtgcagagcctcatgcatcacgg

SEQ ID NO: 25 - DNA, 89 bp, Artificial Sequence
  Oligonucleotide primer
cggccggcaagcccccgctccccgggctctcgcggtcgcacgaggatgcttggcacgtaccccctgtacatacttcccgggcgcccagc

SEQ ID NO: 26 - DNA, 14 bp, Artificial Sequence
  Poly A sequence
aaaaaaaaaaaaaa